(19) United States
(12) Patent Application Publication
(10) Pub. No.: US 2015/0072020 A1
(43) Pub. Date: Mar. 12, 2015

(54) Dexanabinol or a Derivative Thereof for Use in the Treatment of Cancer in Dose Ranges of 2-30 mg/kg

(71) Applicant: E-THERAPEUTICS PLC, Oxfordshire (GB)

(72) Inventors: Malcolm Philip Young, Hexham (GB); Philip McKeown, Gateshead (GB)

(73) Assignee: E-THERAUPEUTICS PLC, Oxfordshire (GB)

(21) Appl. No.: 14/395,541

(22) PCT Filed: Apr. 26, 2013
(86) PCT No.: PCT/GB2013/000183

§ 371 Date: Oct. 20, 2014

(30) Foreign Application Priority Data
Apr. 26, 2012 (GB) 1207305.2

(51) Int. Cl. A61K 31/352; A61K 31/573; A61N 7/00; A61K 31/131; A61K 31/4402; A61K 45/06; A61K 31/341

(52) U.S. Cl. CPC A61K 31/352; A61K 45/06; A61K 31/573; A61K 31/341; A61K 31/131; A61K 31/4402; A61N 7/00
USPC 424/649; 514/454; 549/390; 514/171; 601/2

(57) ABSTRACT

There is described a method of treating cancer in a patient wherein the method comprises the administration of dexanabinol, or a derivative thereof, in an amount of from about 2 mg/kg to about 30 mg/kg, based on the weight of the patient.

### FIELD OF THE INVENTION

The present invention provides medicaments and methods for the treatment of cancer and including a reduction in cell proliferation and/or apoptosis of cancer cells.

More particularly the invention provides the use of certain dosages of dexanabinol, or a derivative thereof, for the treatment of cancers.

### BACKGROUND

Dexanabinol is 1, 1 dimethyl heptyl-(3S, 4S)-7-hydroxy-Δ⁶-tetrahydrocannabinol which is disclosed in U.S. Pat. No. 4,876,276. Dexanabinol is a non psychotropic cannabinoid which has been previously demonstrated to rapidly kill melanoma cells in vitro.

International Patent application WO 2009/007700 describes the use of dexanabinol in the treatment of melanoma cancer cells. The apoptotic effect of dexanabinol is described, but the mechanism of action is not disclosed and was not fully understood at that time. Thus the applicability of the drug for use in other cancer cells other than melanoma was not previously foreseeable. In this previous application it has been disclosed that dexanabinol acts via inhibiting Nuclear Factor Kappa-B (NFκB) in a melanoma cell and thus provides a treatment for melanoma. Furthermore, it has been shown that in melanoma dexanabinol both induces apoptosis and inhibits cell proliferation.

However, the mechanism of action of dexanabinol is more complex than just via binding to NFκB. International Patent application No. WO 2011/030106 describes dexanabinol having an effect on the proteins N-methyl-D-aspartate (NMDA), Cyclooxygenase-2 (COX-2), Tumour Necrosis factor alpha (TNF-a), Nuclear factor-kappa B (NFKB), Cyclin-dependent kinases, e.g. CDK2/A and CDK5/p25, Histone acetyltransferase (HAT) and Farnesyltransferase when administered in a dosage sufficient to achieve a plasma concentration of from 10 to 20 μM.

International Patent application No, WO 03/077832 describes the use of dexanabinol in reducing cancer cell proliferation. Moreover, this decrease in proliferation is described with respect to regulation of inflammation related genes.

However, we have now surprisingly found that that the administration of certain dosages and dosing regimes of dexanabinol, or a derivative thereof, is advantageous and is novel over the prior art.

### SUMMARY OF THE INVENTION

It has been found that the administration of certain dosages of dexanabinol, or a derivative thereof, is an effective cancer therapy, by causing cancer cell apoptosis and/or by reducing cancer cell proliferation.

The known direct and indirect targets of dexanabinol are:

N-methyl-D-Aspartate (NMDA) Receptor

Dexanabinol was originally developed as a neuroprotective agent. Its neuroprotective action was attributed to its ability to block the NMDA receptor. It blocks NMDA-receptors stereospecifically by interacting with a site close to, but distinct from, that of uncompetitive NMDA-receptor antagonists and from the recognition sites of glutamate, glycine, and polyamines. Unlike some other uncompetitive NMDA receptor antagonists, dexanabinol does not produce psychotropic effects and is generally well tolerated in humans.

Cyclooxygenase-2 (COX-2)

Dexanabinol has anti-inflammatory and antioxidative properties unrelated to its capacity to block NMDA receptors. The anti-inflammatory activity was associated with the ability of dexanabinol to reduce the secretion of PGE2 produced by the enzyme cyclooxygenase-2 (COX-2). COX-2 is one of the cyclooxygenase isoforms involved in the metabolism of arachidonic acid (AA) toward prostaglandins (PG) and other eicosanoids, a family of compounds known to exhibit inflammatory properties and known to be involved in inflammation. Most conventional NSAIDs (non-steroidal anti-inflammatory drugs) inhibit COX activity by modifying the enzyme active site thereby preventing the transformation of the AA substrate to PGE2 (Hinz B. et al., J. Pharm. Exp. Ther. 300: 367- 375, 2002). It has been disclosed (WO/2003/077832) that the PGE2 inhibitory activity displayed by dexanabinol does not occur at the level of the COX-2 enzymatic activity, but rather at the level of gene regulation.

Tumour Necrosis Factor Alpha (TNF-a)

Dexanabinol was found to be able to block the production or action of TNF-a. This inhibition most likely occurs at a post-transcriptional level.

Dexanabinol has been found to block the production or action of TNF-a, as disclosed in International Patent applications WO 97/11668 and WO 01/98289. It was postulated that the inhibition of the cytokine occurs at a post-transcriptional stage, since in a model of head injury dexanabinol did not affect the levels of TNF-a mRNA (Shohami E. et al., J. Neuroimmuno. 72: 169-77, 1997).

Human TNF-a is first translated into a 27 kd transmembrane precursor protein, which is cleaved into the secreted 17 kd form by TNF-a converting enzyme (TACE). Based on RT-PCR experiments, Shoshany et al. reported that dexanabinol has no significant effect on TNF-a mRNA whereas it significantly reduced the levels of TACE mRNA, supporting the assumption that the drug acts at the level of secretion inhibition.

Nuclear Factor-Kappa B (NFκB)

There is experimental evidence that Dexanabinol inhibits nuclear factor-kappa B (NFκB) indirectly by inhibiting phosphorylation and degradation of IKB2.

Juttler, E et al. (2004) (Neuropharmacology 47(4):580-92.) provided evidence that dexanabinol inhibits NFκB. Dexanabinol inhibits (1) phosphorylation and degradation of the inhibitor of NF-kappaB IkappaBalpha and translocation of NF-kappaB to the nucleus; dexanabinol reduces (2) the transcriptional activity of NF-kappaB and (3) mRNA accumulation of the NF-kappaB target genes tumour necrosis factor-alpha and interleukin-6 (TNF-alpha and IL-6).

Cyclin-Dependent Kinases: CDK2/A and CDK5/p25

Dexanabinol had no significant direct activity against CDK2 and CDK5, when directly assayed. However, we believe that CDKs are affected indirectly, in circumstances where more of the intracellular network that might mediate such effects remains present.

Histone Acetyltransferase (HAT)

Histone acetyl transferase is a known cancer target. No assay data on whether Dexanabinol has activity against this target, however there is predicted activity at this target, which would thus be beneficial.

Farnesyltransferase

Famesyltransferase is a known cancer target. No assay data on whether Dexanabinol has activity against this target, however there is predicted activity at this target.

Furthermore, dexanabinol, or a derivative thereof, may affect one or more of the following biomarkers:

tumstatin, vascular endothelial growth factor A (VEGF-A), vascular endothelial growth factor D (VEGF-D), soluble vascular endothelial growth factor receptor 1 (sVEGFR1), soluble vascular endothelial growth factor receptor 2 (sVEGFR2), placental growth factor (PlGF), basic fibroblast growth factor (bFGF), stromal cell derived factor 1a (SDF1α), epidermal growth factor (EGF), transforming growth factor beta (TGF-β), platelet derived growth factor (PDGF-AA), platelet derived growth factor (PDGF-AB), platelet derived growth factor (PDGF-BB), angiopoietin-1, thrombospondin-1 and/or interleukin 8 (IL-8).

Dexanabinol has effects at more than one protein that are considered to be important in cancers and in cancer therapy. Some of these effects are direct whereas others are indirect. It is of great importance that dexanabinol has effects at numerous targets and this is makes the compound beneficial in a range of cancers.

Thus, according to a first aspect of the invention there is provided a method of treating cancer in a patient wherein the method comprises the administration of dexanabinol, or a derivative thereof, in an amount of from about 2 mg/kg to about 30 mg/kg, based on the weight of the patient.

Thus, the dosage of dexanabinol, or a derivative thereof, may vary depending upon, inter alia, the severity of the cancer, the nature of the cancer, the sex of the patient, i.e. male or female, etc. and may be about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg or about 30 mg/kg, based on the weight of the patient.

According to a further aspect of the invention there is provided a method of treating cancer in a patient wherein the method comprises the administration of dexanabinol, or a derivative thereof, in an amount sufficient to achieve a plasma concentration of dexanabinol from about 10 to about 100 μM.

Preferably, the method according to this aspect of the invention comprises the administration of dexanabinol, or a derivative thereof, in an amount sufficient to achieve a plasma concentration of dexanabinol from about >20 to about 100 μM.

The dosage of dexanabinol, or a derivative thereof, according to this aspect of the invention may vary depending upon, inter alia, the severity of the cancer, the nature of the cancer, the sex of the patient, i.e. male or female, etc. and may be about 21 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

More specifically, the method may comprise the administration of an effective amount of dexanabinol, or a derivative thereof, as hereinbefore described sufficient to achieve a plasma concentration of dexanabinol, or a derivative thereof, that is maintained for at least 2 hours in the patient.

It will be understood by the person skilled in the art that the aforementioned dosage regime and the frequency of administration may be varied, depending upon, inter alia, the severity of the cancer, the nature of the cancer, the sex of the patient, i.e. male or female, etc. and may be for example, generally based on a dose regime of once weekly, twice weekly, three times weekly, four times weekly, five times weekly, six times weekly, or every day; for one week in a 3 week cycle. Alternatively, the dosage regime may be generally based on a dose regime of once weekly, twice weekly, three times weekly, four times weekly, five times weekly, six times weekly, or every day; for two weeks in a 3 week cycle. Alternatively, the dosage regime may be generally based on a dose regime of once weekly, twice weekly, three times weekly, four times weekly, five times weekly, six times weekly, or every day; for 3 weeks in a 3 week cycle. Alternatively, the dosage regime may be generally based on a dose regime of once weekly, twice weekly, three times weekly, four times weekly, five times weekly, six times weekly, or every day; for one week in a 4 week cycle. Alternatively, the dosage regime may be generally based on a dose regime of once weekly, twice weekly, three times weekly, four times weekly, five times weekly, six times weekly, or every day; for two weeks in a 4 week cycle. Alternatively, the dosage regime may be generally based on a dose regime of once weekly, twice weekly, three times weekly, four times weekly, five times weekly, six times weekly, or every day; for 3 weeks in a 4 week cycle. Alternatively, the dosage regime may be generally based on a dose regime of once weekly, twice weekly, three times weekly, four times weekly, five times weekly, six times weekly, or every day; for 4 weeks in a 4 week cycle.

A course of treatment may comprise of 1, 2, 3, 4, 5, 6 or more cycles. Depending on individual patient response further continuing treatment may be envisioned.

When the dexanabinol, or a derivative thereof, is administered by way of infusion, the duration of the infusion may vary. Thus, the infusion may be administered as an intravenous infusion over a period of 15 minutes, 30 minutes, 45 minutes, 1 hour, 1.5 hours, 2 hours, 2.5 hours, 3 hours, 3.5 hours, 4 hours, 4.5 hours, 5 hours, 5.5 hours, or 6 hours, each treatment day during a cycle.

According to a further aspect of the invention there is provided a therapeutic agent comprising dexanabinol, or a derivative thereof, administrable to a patient in an amount of from about 2 mg/kg to about 30 mg/kg of dexanabinol, or a derivative thereof, based on the weight of the patient.

Thus, the therapeutic comprising dexanabinol, or a derivative thereof, may vary depending upon, inter alia, the severity of the cancer, the nature of the cancer, the sex of the patient, i.e. male or female, etc. and may comprise about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg or about 30 mg/kg, of dexanabinol, or a derivative thereof, based on the weight of the patient.

The therapeutic agent according to this aspect of the invention comprises the administration of dexanabinol, or a derivative thereof, in an amount sufficient to achieve a plasma concentration of dexanabinol from about >20 to about 100 μM.

The dosage of dexanabinol, or a derivative thereof, according to this aspect of the invention may vary depending upon, inter alia, the severity of the cancer, the nature of the cancer, the sex of the patient, i.e. male or female, etc. and may be about 21 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

The invention further provides the use of dexanabinol, or a derivative thereof, in the manufacture of a medicament for the treatment of a cancer wherein the amount of dexanabinol, or a derivative thereof, in the medicament is from about 2 mg/kg to about 30 mg/kg, based on the weight of the patient.

Thus, in the use of dexanabinol, or a derivative thereof, in the manufacture of a medicament as hereinbefore described the amount of dexanabinol, or a derivative thereof, may vary depending upon, inter alia, the severity of the cancer, the nature of the cancer, the sex of the patient, i.e. male or female, etc. and may comprise about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg or about 30 mg/kg, dexanabinol, or a derivative thereof, based on the weight of the patient.

The invention further provides the use of dexanabinol, or a derivative thereof, in the manufacture of a medicament for the treatment of a cancer wherein the amount of dexanabinol, or a derivative thereof, in the medicament is sufficient to achieve a plasma concentration in a patient of dexanabinol of from about >20 to about 100 μM.

The amount of dexanabinol, or a derivative thereof, in the medicament according to this aspect of the invention may vary depending upon, inter alba, the severity of the cancer, the nature of the cancer, the sex of the patient, i.e. male or female, etc. and may be about 21 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

According to a yet further aspect of the invention there is provided a pharmaceutical composition comprising dexanabinol, or a derivative thereof, in admixture with a pharmaceutically acceptable adjuvant, diluent or carrier, wherein the dexanabinol, or a derivative thereof, is in an amount of from about 2 mg/kg to about 30 mg/kg, based on the weight of the patient.

The pharmaceutical composition according to this aspect of the invention may comprise about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg or about 30 mg/kg, dexanabinol, or a derivative thereof, based on the weight of the patient.

Further according to this aspect of the invention there is provided a pharmaceutical composition comprising dexanabinol, or a derivative thereof, in admixture with a pharmaceutically acceptable, adjuvant, diluent or carrier, wherein the amount, of dexanabinol, or a derivative thereof, is sufficient to achieve a plasma concentration in a patient of dexanabinol of from about >20 to about 100 μM.

The amount of dexanabinol, or a derivative thereof, in the pharmaceutical composition according to this aspect of the invention may vary depending upon, inter alia, the severity of the cancer, the nature of the cancer, the sex of the patient, i.e. male or female, etc. and may be about 21 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

The pharmaceutical composition according to this aspect of the invention may comprise from about 200 mg to about 2,000 mg of dexanabinol, or a derivative thereof, in admixture with a pharmaceutically acceptable adjuvant, diluent or carrier.

The amount of dexanabinol, or a derivative thereof, in the pharmaceutical composition according to this aspect of the invention may vary depending upon, inter alia, the severity of the cancer, the nature of the cancer, the sex of the patient, i.e. male or female, etc. and may be about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 1,050 mg, about 1,100 mg, about 1,150 mg, about 1,200 mg, about 1,250 mg, about 1,300 mg, about 1,350 mg, about 1,400 mg, about 1,450 mg, about 1,500 mg, about 1,550 mg, about 1,600 mg, about 1,650 mg, about 1,700 mg, about 1,750 mg, about 1,800 mg, about 1,850 mg, about 1,900 mg, about 1,950 mg or about 2,000 mg.

It will be understood that the dexanabinol, or a derivative thereof, may have an effect on the proteins N-methyl-D-aspartate (NMDA), Cyclooxygenase-2 (COX-2), Tumour Necrosis factor alpha (TNF-a), Nuclear factor-kappa B (NFiκB), Cyclin-dependent kinases, e.g. CDK2/A and CDK5/p25, Histone acetyltransferase (HAT) and Farnesyltransferase, simultaneously, sequentially or separately.

In the treatment of cancer according to the present invention the cancer may be one or more of adenoma, astrocytoma, anal cancer, benign tumours, blastoma, brain cancer, brain metastases, breast cancer, cancer (malignant neoplasm), basal cell carcinoma, bile duct cancer, Burkitt lymphoma, cervical cancer, colon cancer, colorectal cancer, endometrial cancer, epithelial carcinoma, gall bladder cancer, gastric carcinoma, germ cell tumours, glioblastoma multiforme, glioblastoma, glioma, head and neck cancer, hepatocellular carcinoma, high grade gliomas, intrahepatic bile duct cancer, laryngeal cancer, leukaemia, (acute. lymphoblastic leukemia (ALL), acute myeloid leukemia (AML), chronic lymphocytic leukemia (CLL) and chronic myeloid leukemia CML), lip cancer, liver cancer, lymphoma, melanoma, menigioma, mesothelioma, metastatic cancers, myeloma, non-small cell lung cancer, oesophageal cancer, oral cancer, osteosarcoma, ovarian cancer, pancreatic cancer, pharyngeal cancer, pituitary tumours, primary cancer, prostate cancer, renal cancer, sarcoma, small cell lung cancer, stomach cancer, testicular cancer, thyroid cancer, thyroid carcinoma, urinary bladder cancer and uterine cancer. In particular, the cancer may be one or more of brain metastases and high grade gliomas.

Brain Metastases

Brain metastases are the most common intracranial neoplasm, occurring in 10-30% of cancer patients, and are a significant cause of morbidity and mortality. Among adults, lung cancer accounts for approximately half of these cases. Other primary disease that is metastatic to the brain includes breast cancer (15-20% of cases), melanoma (10%), renal cancer, colorectal cancer, lymphoma, and tumours of unknown primary [Norden, 2005]. The incidence of brain metastases has been increasing for a number of reasons, including longer survival of patients with metastatic primary disease from more effective systemic therapy and enhanced detection. Current treatment modalities include surgery, stereotactic radio surgery (SRS), whole brain radiation (WBRT), and chemotherapy. For metastases that reoccur, there is no FDA approved treatment besides radiation therapy. Based on various prognostic factors, median survival of patients with brain metastases ranges from 2.3 to 13.5 months [Gaspar, 2000].

High Grade Gliomas

Primary malignant gliomas, glioblastoma (GBM) in particular, represent the second most common intracranial neoplasm. Standard of care results in a median survival of 14 months. Despite advances in treatment for newly diagnosed glioma patients, essentially all patients will experience disease recurrence. For patients with recurrent disease, conventional chemotherapy is generally ineffective with response rates <20%. Like metastatic cancers to the brain, there is high frequency of diffuse and leptomeningeal metastases from primary gliomas. Recent genome-wide studies have confirmed that GBM is a heterogeneous group of diseases that can be subclassified by shared genetic aberrations [Parsons, 2008; McLendon, 2008]. The implication is that, in part, the underlying genetics may determine responsiveness to treatments and thus allow us to personalize therapy. With dismal prognoses and few effective treatments, clearly new therapies are critically needed for brain cancer patients.

Furthermore, the cancer may selected from one or more of pancreatic carcinoma, glioblastoma, gastric carcinoma, oesophageal carcinoma, ovarian carcinoma, renal carcinoma and thyroid carcinoma.

Thus, the dexanabinol, or a derivative thereof will be a therapeutically effective amount. According to the present invention, a therapeutically effective amount may mean au effective amount for apoptosis of cancer cells, inhibition of cancer cell proliferation, inhibition of tumourigenesis and/or induction of cytotoxicity.

The method or use of the invention may comprise the administration of a therapeutically effective amount of dexanabinol, or a derivative thereof, sufficient to inhibit tumourigenesis of a cancer cell.

Alternatively or in addition the method or use of the invention may comprise the administration of a therapeutically effective amount dexanabinol, or a derivative thereof, sufficient to induce cytotoxicity in the cancer cell.

Alternatively or in addition the method or use of the invention may comprise the administration of a therapeutically effective amount dexanabinol, or a derivative thereof, sufficient to induce apoptosis of the cancer cell.

The present invention contemplates that the cancer cells may be premalignant, malignant, primary, metastatic or multidrug-resistant

Alternatively, the treatment of the cancer may comprise the inhibition of tumourigenesis of a cancer cell by contacting the cell with an effective amount of dexanabinol, or a derivative thereof. Inhibition of tumourigenesis may also include inducing cytotoxicity and/or apoptosis in the cancer cell.

Furthermore the method or use of the invention as hereinbefore described is advantageous because, inter alia, it shows reduced toxicity, reduced side effects and/or reduced resistance when compared to those chemotherapeutic agents currently employed.

It is further contemplated that a second therapy may be provided in combination with dexanabinol, or a derivative thereof, as hereinbefore described, to a cancer cell for treatment and/or prevention of the cancer. The second therapeutic agent may comprise a chemotherapeutic agent, immunotherapeutic agent, gene therapy or radio therapeutic agent. When a second therapeutic agent is included in the treatment according to the invention, the second therapeutic agent may be administered with the dexanabinol, or a derivative thereof, separately, simultaneously or sequentially.

Although a variety of second or additional therapeutic agents may be used in conjunction with dexanabinol, or a derivative thereof, preferably, the second or additional therapeutic agent may be selected from the group consisting of: a chemotherapeutic agent, an immunotherapeutic agent, a gene therapy agent, and a radiotherapeutic agent.

According to a further aspect of the invention, dexanabinol, or a derivative thereof, may be administered in combination, separately, simultaneously or sequentially, with a second therapy wherein the second therapy is selected from the group consisting of one or more of a chemotherapeutic agent; an alkylating agent, such as carmustine or temozolamide; a mitotic inhibitor, such as taxanes, (e.g. paclitaxol or docetaxol) or vinca alkaloids (e.g. vinblastine, vincristine, vindestine or vinorelbine); platinum derived compounds (e.g. carboplatin, cisplatin, nedaplatin, oxaliplatin, triplatin tetranitrate or satraplatin); dihydrofolate reductase inhibitors (e.g. aminopterin, methotrexate, pemetrexed or pralatrexate); a DNA polymerase inhibitor (e.g.

cytarabine); a ribonucleotide reductase inhibitor (e.g. gemcitabine); a thymidylate synthase inhibitors (e.g. fluorouracil, capecitabine, tegafur, carmofur or floxuridine); aspirin; a non-steroidal anti-inflammatory agent (e.g. ibuprofen); a steroidal anti inflammatory agent (e.g. a corticosteroid, such as, prednisolone or cortisol); a non-drug oncology therapeutic agent; radiotherapy; tumour embolisation; surgery; and ultrasound.

Thus, according to this aspect of the invention there is provided dexanabinol, or a derivative thereof, in combination with at least a second thereape3utic agent. More specifically, the invention provides:

dexanabinol, or a derivative thereof, in combination with alkylating agents such as carmustine or temozolamide. separately, simultaneously or sequentially;

dexanabinol, or a derivative thereof, in combination with mitotic inhibitors such as taxanes, (e.g. paclitaxol or docetaxol), vinca alkaloids (e.g. vinblastine, vincristine, vindestine, or vinorelbine) separately, simultaneously or sequentially;

dexanabinol, or a derivative thereof, in combination with platinum derived compounds (e.g. carboplatin, cisplatin, nedaplatin, oxaliplatin, triplatin tetranitrate satraplatin) separately, simultaneously or sequentially;

dexanabinol, or a derivative thereof, in combination with dihydrofolate reductase inhibitors (e.g. aminopterin, methotrexate, pemetrexed or pralatrexate) separately, simultaneously or sequentially;

dexanabinol, or a derivative thereof, in combination with DNA polymerase inhibitor (e.g. cytarabine) separately, simultaneously or sequentially;

dexanabinol, or a derivative thereof, in combination with ribonucleotide reductase inhibitor (e.g. gemcitabine) separately, simultaneously or sequentially;

dexanabinol, or a derivative thereof, in combination with thymidylate synthase inhibitors (e.g. fluorouracil capecitabinetegafur carmofur floxuridine) separately, simultaneously or sequentially;

dexanabinol, or a derivative thereof, in combination with aspirin separately, simultaneously or sequentially;

dexanabinol, or a derivative thereof, in combination with non steroidal anti inflammatory agents (e.g. ibuprofen) separately, simultaneously or sequentially;

dexanabinol, or a derivative thereof, in combination with steroidal anti inflammatory agents (e.g. corticosteroids such as prednisolone or cortisol) separately, simultaneously or sequentially;

dexanabinol, or a derivative thereof, in combination with non drug oncology therapeutic agent separately, simultaneously or sequentially;

dexanabinol, or a derivative thereof, in combination with radiotherapy separately, simultaneously or sequentially;

dexanabinol, or a derivative thereof, in combination with tumour embolisation separately, simultaneously or sequentially;

dexanabinol, or a derivative thereof, in combination with surgery separately, simultaneously or sequentially; and/or

dexanabinol, or a derivative thereof, in combination with ultrasound separately, simultaneously or sequentially.

The term “derivative” used herein shall include any conventionally known derivatives of dexanabinol, such as, inter alfa, solvates. It may be convenient or desirable to prepare, purify, and/or handle a corresponding solvate of the compound described herein, which may be used in any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate. The term derivative shall especially include a salt. Suitable salts of dexanabinol are well known and are described in the prior art. Salts of organic and inorganic acids and bases that may be used to make pharmaceutically acceptable salts. Such acids include, without limitation, hydrofluoric, hydrochloric, hydrobromic, hydroiodic, sulphuric, nitric, phosphoric, citric, succinic, maleic, and palmitic acids. The bases include such compounds as sodium and ammonium hydroxides. Those skilled in the art are familiar with quaternising agents that can be used to make pharmaceutically acceptable quaternary ammonium derivatives of dexanabinol. These include without limitation methyl and ethyl iodides and sulphates.

Dexanabinol and derivatives and/or combinations thereof are known per se and may be prepared using methods known to the person skilled in the art or may be obtained commercially. In particular, dexanabinol and methods for its preparation are disclosed in U.S. Pat. No. 4,876,276.

The dexanabinol, or a derivative thereof, may be administered in a variety of ways by and by any conventional and appropriate route, depending upon, inter alia, the nature of the cancer to be treated. Thus, the dexanabinol, or a derivative thereof, may be administered topically, transdermally, subcutaneously, intravenously intramuscularly, orally, parenterally, intrathecally, rectally or intranasally.

We especially provide the method or use of dexanabinol, or a derivative thereof, as hereinbefore described which comprises the intravenous (IV) administration of dexanabinol, or a derivative thereof.

For intravenous administration the pharmaceutical composition of the invention as hereinbefore described may comprise a solvent, such as an alcohol, e.g. ethanol, and a surfactant, e.g. a non-ionic surfactant. A preferred non-ionic surfactant is a polyethoxylated castor oil, such as Cremophor EL® (polyethoxylated 35 castor oil) available from BASF. The pharmaceutical composition of the invention may also include an antioxidant, such as, edetic acid (EDTA-acid) and/or vitamin E (DL-α-tocopherol).

Dexanabinol is highly lipophilic and therefore the method of treatment of the present invention may also include a pre-medication step prior to the administration of a dexanabinol therapy. According to the present invention dexanabinol; or a derivative thereof, may, for example, be dissolved in a co-solvent mixture of Cremophor® and ethanol. Therefore, a pre-medication may be administered approximately 30 minutes prior to administration of each dexanabinol intravenous infusion of dexanabinol, or a derivative thereof, following standard institutional practices for prophylaxis of hypersensitivity reactions with Cremophor®-containing anti-cancer agents.

Thus, by way of example, such a pre-medication may consist of one or more of:
an anti-inflammatory/immunosuppressant, such as a steroid, e.g. dexamethasone (IV);
a histamine H₂-receptor antagonist, such as, ranitidine (IV), cimetidine (IV), etc.; and
an antihistamine, such as, diphenhydramine (IV) or chlorphenamine (IV).

When the method of the invention includes a pre-treatment as hereinbefore described, the amount of pre-treatment may vary, depending upon, inter alia, the amount of dexanabinol, or a derivative thereof, to be administered, the nature of the pre-treatment, etc. However, the pre-treatment may desirably comprise one or more of
from about 1 to about 50 mg of anti-inflammatory/immunosuppressant, such as a steroid, e.g. 10 mg or 20 mg dexamethasone (IV);
from about 10 to about 100 mg of a histamine H₂-receptor antagonist, such as, 50 mg ranitidine (IV) or 50 mg cimetidine (IV), etc.; and
from about 1 to about 100 mg an antihistamine, such as, 50 mg diphenhydramine (IV) or 10 mg chlorphenamine (N).

According to a yet further aspect f the invention there is provided a kit comprising:
a pharmaceutical composition as hereinbefore described; and
a pre-treatment as hereinbefore described.

Thus, in the use, method and/or composition of the invention of the compound may be put up as a tablet, capsule, dragee, suppository, suspension, solution, injection, e.g. intravenously, intramuscularly or intraperitoneally, implant, a topical, e.g. transdermal, preparation such as a gel, cream, ointment, aerosol or a polymer system, or an inhalation form, e.g. an aerosol or a powder formulation.

Compositions suitable for oral administration include tablets, capsules, dragees, liquid suspensions, solutions and syrups;

Compositions suitable for topical administration to the skin include creams, e.g. oil-in-water emulsions, water-in-oil emulsions, ointments, gels, lotions, unguents, emollients, colloidal dispersions, suspensions, emulsions, oils, sprays, foams, mousses, and the like. Compositions suitable for topical application may also include, for example, liposomal carriers made up of lipids or special detergents.

Examples of other adjuvants, diluents or carriers are:

for tablets and dragees—fillers, e.g. lactose, starch, microcrystalline cellulose, talc and stearic acid; lubricants/glidants, e.g. magnesium stearate and colloidal silicon dioxide; disintegrants, e.g. sodium starch glycolate and sodium carboxymethylcellulose;

for capsules—pregelatinised starch or lactose;

for oral or injectable solutions or enemas—water, glycols, alcohols, glycerine, vegetable oils;

for suppositories—natural or hardened oils or waxes.

It may be possible to administer the compound or derivatives and/or combination thereof or any combined regime as described above, transdermally via, for example, a transdermal delivery device or a suitable vehicle or, e.g. in an ointment base, which may be incorporated into a patch for controlled delivery. Such devices are advantageous, as they may allow a prolonged period of treatment relative to, for example, an oral or intravenous medicament.

Examples of transdermal delivery devices may include, for example, a patch, dressing, bandage or plaster adapted to release a compound or substance through the skin of a patient. A person of skill in the art would be familiar with the materials and techniques which may be used to transdermally deliver a compound or substance and exemplary transdermal delivery devices are provided by GB2185187, U.S. Pat. No. 3,249,109, U.S. Pat. No. 3,598,122, U.S. Pat. No. 4,144,317, U.S. Pat. No. 4,262,003 and U.S. Pat. No. 4,307,717.

The invention will now be illustrated by way of example only.

### DETAILED DESCRIPTION OF THE INVENTION

### Example 1

Dose Form/Formulation:

Dexanabinol Drug Product is a clear, slightly yellow solution formulated for intravenous (IV) administration as a 5% (w/v) concentrate in an ethanol and Cremophor® EL (polyoxyl 35 castor oil) co-solvent vehicle, with edetic acid (EDTA-acid) and vitamin E (DL-a-tocopherol) as antioxidants.

Dexanabinol Drug Product is diluted with sterile 0.9% sodium chloride to a final concentration of 0.2-4 mg/L prior to administration.

| | | | | Quantity per unit |
|---|---|---|---|---|
| | | | | mg |
| Component/Grade | Function | mg/mL | mg/g | 4.7 mL fill volume |
| Dexanabinol | API | 50.0 | 51.5 | 235.0 |
| Ethanol Absolute, BP | Solvent | 265.0 | 237.2 | 1245.5 |
| Cremophor EL USP | Solvent | 650.0 | 670.0 | 3055.0 |
| (polyoxyl 35 castor | | | | |
| oil) | | | | |
| Edetic acid USP | Chelating | 0.1 | 0.1 | 0.47 |
| | agent | | | |
| DL-α-Tocopherol | Solubility | 5.0 | 5.2 | 23.03 |
| USP | | | | |

### Example 2

Pre-Medication

Dexanabinol is highly lipophilic. It is dissolved in a co-solvent mixture of Cremophor® and ethanol; therefore the following pre-medications will be given approximately 30 minutes prior to administration of each dexanabinol infusion, following standard institutional practices for prophylaxis of hypersensitivity reactions with Cremophor®-containing anti-cancer agents:

The pre-medication comprises:
10 mg dexamethasone IV;
50 mg ranitidine IV (or equivalent); and
50 mg diphenhydramine IV.

OR
20 mg dexamethasone IV;
50 mg ranitidine IV (or equivalent); and
10 mg chlorphenamine TV

### Example 3

A Phase 1, Pharmacokinetically-Guided, Dose Escalation Study to Assess the Safety and Tolerability of Dexanabinol in Patients With Advanced Solid Tumours

This is a Phase 1, open-label, dose escalation study of the safety, tolerability, and pharmacokinetics (PK) of Dexanabinol in patients with advanced solid tumours. Eligible participants will be enrolled in 3-patient cohorts treated with Dexanabinol, formulated in Cremophor®/ethanol, given as a 3 hour infusion on Days 1, 8 and 15 of a 3-week cycle, while being monitored for safety and DLTs.

Primary Outcome Measures:
Maximum Tolerated Dose (MTD) [Time Frame: Each patient will be followed for 22 days ]
Patients will be sequentially assigned to increasing doses of Dexanabinol, to establish the MTD (highest dose it is safe to give patients) or alternatively the Maximum Administered Dose (MAD).
3 patients will be enrolled to a cohort to assess each dose level. Dose escalation to a cohort of 3 new patients will occur when all patients in the previous cohort have completed the first cycle i.e. the first 3 doses followed by observation through to Day 22, and no Dose Limiting Toxicity (DLT) has occurred.
DLTs will be graded for severity based on the NCI Common Terminology Criteria version 4.03

Secondary Outcome Measures:
Area Under Curve (AUC) of Dexanabinol and Cremophor [Time Frame: Cycle1—Day 1 and 8: pre-dose (0 h); 1, 2, 3 h post start of infusion; 5, 10, 15, 30 min post-end infusion; 1, 2, 3, 4, 6, 8, 10 and 24 h post-end infusion. Day 15: immediately prior to infusion and at the end of infusion.]
Maximum Concentration (Cmax) of Dexanabinol and Cremophor [Time Frame: Cycle1—Day 1 and 8: pre-dose (0 h); 1, 2, 3 h post start of infusion; 5, 10, 15, 30 min post-end infusion; 1, 2, 3, 4, 6, 8, 10 and 24 h post-end infusion. Day 15: immediately prior to infusion and at the end of infusion.]
Minimum Concentration (Cmin) of Dexanabinol and Cremophor [Time Frame: Cycle 1—Day 1 and 8: pre-dose (0 h); 1, 2, 3 h post start of infusion; 5, 10, 15, 30 min post-end infusion; 1, 2, 3, 4, 6, 8, 10 and 24 h post-end infusion. Day 15: immediately prior to infusion and at the end of infusion.]
Number of adverse events (AEs) [Time Frame: 30 +/−3 days from the end of the last infusion.]
AEs will be graded according to the NCI CTCAE v4.03 for cancer clinical trials.
Tumour response [Time Frame: At Screening and after every 2 cycles of treatment (+/−1 week) ][Designated as safety issue: No.]
Tumour response evaluation using RECIST 1.1. (Assessment by CT scan or MRI). An additional scan will be performed to confirm a Complete Response
(CR) or Partial Response (PR). Tumour markers may be evaluated where appropriate.

### Example 4

A Phase I, Sequential Cohort, Open-Label, Dose-escalation Study of the Safety and CNS Pharmacokinetics of Dexanabinol in Patients with Brain Cancer

This is an open-label, single institution, Phase I 3+3 dose escalation study of dexanabinol in patients with brain cancer having failed prior therapy. Treatment cycle (28 days) will consist of dexanabinol administered intravenously over three hours once weekly on Days 1, 8, 15, and 22.

Primary Objective

To determine the safety and/or tolerability and the recommended phase 2 dose (RP2D) of intravenously administered dexanabinol in patients with recurrent gliomas or brain metastases.

Secondary Objectives
To assess the exposure to dexanabinol in the cerebrospinal fluid (CSF) and serum.
To assess preliminary evidence of response to dexanabinol as measured by overall survival, progression free survival and objective tumour response.
To explore the association between molecular phenotype and patient response and survival.
To explore disease-related patient-reported outcomes using the FACT-Br instrument.

1. A method of treating cancer in a patient wherein the method comprises the administration of dexanabinol, or a derivative thereof, in an amount of from about 2 mg/kg to about 30 mg/kg, based on the weight of the patient.

2. (canceled)

3. A method of treating cancer in a patient wherein the method comprises the administration of dexanabinol, or a derivative thereof, in an amount sufficient to achieve a plasma concentration of dexanabinol from about 10 to about 100 μM.

4. (canceled)

5. (canceled)

6. A method according to claim 1 wherein the dosage of dexanabinol, or a derivative thereof, is sufficient to achieve a plasma concentration of dexanabinol, or a derivative thereof, that is maintained for at least 2 hours in the patient.

7. A method according to claim 1 wherein the dose regime comprises administration once weekly, twice weekly, three times weekly, four times weekly, five times weekly, six times weekly, or every day; for one week in a 3 week cycle.

8. (canceled)

9. (canceled)

10. A method according to claim 1 wherein the dose regime comprises administration once weekly, twice weekly, three times weekly, four times weekly, five times weekly, six times weekly, or every day; for one week in a 4 week cycle.

11. (canceled)

12. (canceled)

13. A method according to claim 1 wherein the dose regime comprises administration once weekly, twice weekly, three times weekly, four times weekly, five times weekly, six times weekly, or every day.

14. A method according to claim 7 wherein the dose regime comprises administration a course of treatment comprising of 1, 2, 3, 4, 5, 6 or more cycles.

15. A method according to claim 1 wherein the method comprises administration by infusion.

16. (canceled)

17. A method according to claim 15 wherein the infusion is administered over a period of 15 minutes, 30 minutes, 45 minutes, 1 hour, 1.5 hours, 2 hours, 2.5 hours, 3 hours, 3.5 hours, 4 hours, 4.5 hours, 5 hours, 5.5 hours, or 6 hours, each treatment day during a cycle.

18. A method according to claim 1 wherein the cancer is selected from one or more of adenoma, astrocytoma, anal cancer, benign tumours, blastoma, brain cancer, brain metastases, breast cancer, cancer (malignant neoplasm), basal cell carcinoma, bile duct cancer, Burkitt lymphoma, cervical cancer, colon cancer, colorectal cancer, endometrial cancer, epithelial carcinoma, gall bladder cancer, gastric carcinoma, germ cell tumours, glioblastoma multiforme, glioblastoma, glioma, head and neck cancer, hepatocellular carcinoma, high grade gliomas, intrahepatic bile duct cancer, laryngeal cancer, leukaemia (ALL, AML, CLL, CML), lip cancer, myeloma, liver cancer, lymphoma, melanoma, menigioma, mesothelioma, metastatic cancers, myeloma, non-small cell lung cancer, oesophageal cancer, oral cancer, osteosarcoma, ovarian cancer, pancreatic cancer, pharyngeal cancer, pituitary tumours, primary cancer, prostate cancer, renal cancer, sarcoma, small cell lung cancer, stomach cancer, testicular cancer, thyroid cancer, thyroid carcinoma, urinary bladder cancer and uterine cancer.

19. A method according to claim 18 wherein the cancer is selected from one or more of brain metastases and high grade gliomas.

20. A method according to claim 1 wherein the method includes a second therapy, separately, simultaneously or sequentially.

21. A method according to claim 20 wherein the second therapeutic agent is selected from one or more of a chemotherapeutic agent, immunotherapeutic agent, gene therapy and radio therapeutic agent.

22. A method according to claim 20 wherein the second therapy is selected from the group consisting of one or more of a chemotherapeutic agent; an alkylating agent, such as carmustine or temozolamide; a mitotic inhibitor, such as taxanes, (e.g. paclitaxol or docetaxol) or vinca alkaloids (e.g. vinblastine, vincristine, vindestine or vinorelbine);
platinum derived compounds (e.g. carboplatin, cisplatin, nedaplatin, oxaliplatin, triplatin tetranitrate or satraplatin); dihydrofolate reductase inhibitors (e g aminopterin, methotrexate, pemetrexed or pralatrexate); a DNA polymerase inhibitor (e.g. cytarabine); a ribonucleotide reductase inhibitor (e.g. gemcitabine); a thymidylate synthase inhibitors (e.g. fluorouracil, capecitabine, tegafur, carmofur or floxuridine); aspirin; a non-steroidal anti-inflammatory agent (e.g. ibuprofen); a steroidal anti inflammatory agent (e.g. a corticosteroid, such as, prednisolone or cortisol); a non-drug oncology therapeutic agent; radiotherapy; tumour embolisation; surgery; and ultrasound.

23. A method according to claim 1 wherein the method includes the administration of a pre-treatment.

24. A method according to claim 23 wherein the pre-treatment comprises the administration of one or more of:
an anti-inflammatory/immunosuppressant;
a histamine H₂-receptor antagonist; and
an antihistamine.

25-31. (canceled)

32. A therapeutic agent comprising dexanabinol, or a derivative thereof, administrable to a patient in an amount of from about 2 mg/kg to about 30 mg/kg, of dexanabinol, or a derivative thereof, based on the weight of the patient.

33-55. (canceled)

56. A pharmaceutical composition comprising dexanabinol, or a derivative thereof, in admixture with a pharmaceutically acceptable adjuvant, diluent or carrier, wherein the dexanabinol, or a derivative thereof, is present in an amount of from about 2 mg/kg to about 30 mg/kg, based on the weight of the patient.

57-75. (canceled)
